(19) United States
(12) Patent Application Publication
(10) Pub. No.: US 2026/0364383 A1
(43) Pub. Date: Nov. 19, 2026

(54) ULTRASOUND DEVICE FOR ACCELERATING LYMPHATIC CIRCULATION AND METHOD OF OPERATING THE ULTRASOUND DEVICE

(71) Applicant: Research & Business Foundation SUNGKYUNKWAN UNIVERSITY, Suwon-si (KR)

(72) Inventors: Jinhyoung PARK, Suwon-si (KR); Taewon CHOI, Suwon-si (KR); Seoyun CHANG, Suwon-si (KR)

(73) Assignee: Research & Business Foundation SUNGKYUNKWAN UNIVERSITY, Suwon-si (KR)

(21) Appl. No.: 19/286,406

(22) Filed: Jul. 31, 2025

(30) Foreign Application Priority Data
May 19, 2025 (KR) 10-2025-0064726

(51) Int. Cl. A61N 7/00

(52) U.S. Cl. CPC A61N 7/00; A61N 2007/0082

(57) ABSTRACT

An ultrasound device for accelerating lymphatic circulation and a method of operating the ultrasound device are provided. The ultrasound device includes an ultrasound generator, an ultrasound converter, a motor, and at least one processor operationally connected to the ultrasound generator, the ultrasound converter, and the motor, wherein the at least one processor is configured to determine a time at which the ultrasound beam is radiated from the ultrasound converter, control the ultrasound converter to radiate the ultrasound beam based on the time at which the ultrasound beam is radiated, move the ultrasound converter to correspond to a sweeping operation of moving and radiating the ultrasound beam through the motor, and convert the output characteristics of the ultrasound beam through the ultrasound converter to correspond to a focusing position of the ultrasound beam during the sweeping operation.

### CROSS-REFERENCE TO RELATED APPLICATION

This application is based on and claims priority under 35 U.S.C. §119 to Korean Patent Application No. 10-2025-0064726, filed on May 19, 2025, in the Korean Intellectual Property Office, the disclosure of which is incorporated by reference herein in its entirety.

### BACKGROUND

### 1. Field

The disclosure relates to an ultrasound device for accelerating lymphatic circulation and a method of operating the ultrasound device. Specifically, the disclosure relates to a technology for continuously radiating an ultrasound beam while moving an ultrasound focusing point.

This research was supported by the Samsung Future Technology Promotion Project (Project No.: SRFC-IT2201-04).

### 2. Description of the Related Art

Lymph circulates inside the human body. Lymph may circulate through lymphatic vessels formed along the tissues of the human body. Lymph may supply nutrients to the tissues. Lymph may receive waste materials from the tissues. Lymph may remove waste materials from the tissues by discharging the waste materials into the veins through inter-tissue circulation and lymphatic circulation.

Various methods have been proposed to accelerate lymphatic circulation in order to remove waste materials generated from the tissues of the human body. In particular, a method using an ultrasound beam may directly stimulate the interior of the tissue without using chemical methods and accelerate lymphatic circulation. Recently, a method of scanning an ultrasound beam across a tissue has been proposed.

However, when the ultrasound beam is scanned on the entire tissue, the lymphatic circulation acceleration effect may be small or side effects may occur due to the ultrasound beam. Accordingly, a new technology for radiating an ultrasound beam to increase the acceleration of lymphatic circulation while minimizing side effects, and an ultrasound device for implementing the technology, are needed.

### SUMMARY

Additional aspects will be set forth in part in the description which follows and, in part, will be apparent from the description, or may be learned by practice of the presented embodiments of the disclosure.

According to an aspect of the disclosure, an ultrasound device for accelerating lymphatic circulation includes an ultrasound generator configured to generate an ultrasound beam, an ultrasound converter configured to radiate the ultrasound beam generated by the ultrasound generator and convert output characteristics of the ultrasound beam, a motor configured to move the ultrasound converter, and at least one processor operationally connected to the ultrasound generator, the ultrasound converter, and the motor, wherein the at least one processor is configured to determine a time at which the ultrasound beam is radiated from the ultrasound converter, control the ultrasound converter to radiate the ultrasound beam based on the time at which the ultrasound beam is radiated, move the ultrasound converter to correspond to a sweeping operation of moving and radiating the ultrasound beam through the motor, and convert the output characteristics of the ultrasound beam through the ultrasound converter to correspond to a focusing position of the ultrasound beam during the sweeping operation.

According to an aspect of the disclosure, an ultrasound device for accelerating lymphatic circulation includes a multi-channel ultrasound generator configured to generate an ultrasound beam, an array ultrasound converter configured to radiate the ultrasound beam generated by the multi-channel ultrasound generator and simulate a movement of the ultrasound beam, a memory storing position information including a position at which the ultrasound beam is radiated, and at least one processor operationally connected to the multi-channel ultrasound generator, the array ultrasound converter, and the memory, wherein the at least one processor is configured to determine a time at which the ultrasound beam is radiated from the array ultrasound converter, control the array ultrasound converter to radiate the ultrasound beam based on the time at which the ultrasound beam is radiated, move a focusing position of the ultrasound beam through the array ultrasound converter to correspond to a sweeping operation of the ultrasound beam or the position information stored in the memory, and simulate a reciprocating motion of the ultrasound beam through the array ultrasound converter to correspond to the focusing position of the ultrasound beam during the sweeping operation.

According to an aspect of the disclosure, a method of operating an ultrasound device for accelerating lymphatic circulation includes determining, by the ultrasound device, a time at which an ultrasound beam is radiated from the ultrasound device, radiating, by the ultrasound device, the ultrasound beam based on the time at which the ultrasound beam is radiated, moving, by the ultrasound device, a focusing position of the ultrasound beam to correspond to a sweeping operation of the ultrasound beam or a position at which the ultrasound beam is radiated, and converting, by the ultrasound device, output characteristics of the ultrasound beam or simulating a reciprocating motion of the ultrasound beam to correspond to the focusing position of the ultrasound beam during the sweeping operation.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram illustrating an operation concept of an ultrasound device for accelerating lymphatic circulation according to an embodiment;

FIG. 2 is a block diagram illustrating an ultrasound device according to an embodiment;

FIG. 3 is a block diagram illustrating a processor according to an embodiment;

FIG. 4 is a block diagram illustrating an ultrasound device according to an embodiment;

FIG. 5 is a diagram illustrating a method of operating an ultrasound converter according to an embodiment;

FIG. 6 is a diagram illustrating that an ultrasound converter is in contact with a subject according to an embodiment;

FIG. 7 is a diagram illustrating a method of moving an ultrasound converter according to an embodiment;

FIG. 8 is a graph illustrating a distribution of longitudinal wave pressure according to a moving direction of an ultrasound converter according to an embodiment;

FIG. 9 is a block diagram illustrating an ultrasound device according to an embodiment;

FIG. 10 is a block diagram illustrating a multi-channel ultrasound generator according to an embodiment;

FIG. 11 is a diagram illustrating an array ultrasound converter according to an embodiment;

FIG. 12 is a diagram illustrating sweeping of an array ultrasound converter according to an embodiment;

FIG. 13A is a diagram illustrating a sweeping form of an ultrasound device according to an embodiment;

FIG. 13B is a diagram illustrating a sweeping form of an ultrasound device according to an embodiment;

FIG. 13C is a diagram illustrating a sweeping form of an ultrasound device according to an embodiment;

FIG. 14 is a flowchart illustrating a method of operating ultrasound devices according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term "and/or" includes any and all combinations of one or more of the associated listed items. Expressions such as "at least one of," when preceding a list of elements, modify the entire list of elements and do not modify the individual elements of the list.

Various embodiment of the document and terms used therein are not intended to limit the disclosure to particular modes of practice, and it is to be appreciated that various modifications, equivalents, and/or alternatives that do not depart from the spirit and technical scope of the disclosure are encompassed in the disclosure.

Throughout the drawings, similar or relevant parts are referenced with similar reference numerals.

The singular form of the noun corresponding to an item may include one item or a plurality of items, unless the relevant context clearly dictates otherwise.

In the disclosure, the expressions such as "A or B," "at least one of A and/or B," or "at least one or more of A and/or B" may include all available combinations of items listed together. For example, the expressions such as "A or B," "at least one of A and B," or "at least one of A or B" may signify all cases of including at least one A, including at least one B, or including both of at least one A and at least one B.

The term "and/or" includes any and all combinations of one or more of the associated listed items.

Terms such as "1^{st}" and "2^{nd}" or "first" and "second" are used herein merely to distinguish one component from another component and do not limit the components in another aspect (e.g., order or importance).

When a component (e.g., a first component) is referred to as being "coupled" or "connected" to another component (e.g., a second component), with or without the terms "functionally" or "communicatively," it means that the component may be connected to the other component directly (e.g., wired), wirelessly, or via a third component.

Terms such as "include" or "comprise" may be construed to denote a certain characteristic, number, step, operation, component, or a combination thereof, but may not be construed to exclude the existence of or a possibility of addition of one or more other characteristics, numbers, steps, operations, components, or combinations thereof.

When a component is "connected," "coupled," "supported," or "in contact" with another component, this includes not only cases where the components are directly connected to, coupled to, supported by, or in contact with each other, but also cases where the components are indirectly connected to, coupled to, supported by, or in contact with each other through a third component.

When a component is referred to as being "on" another component, the component may be directly on another component or between two intervening components.

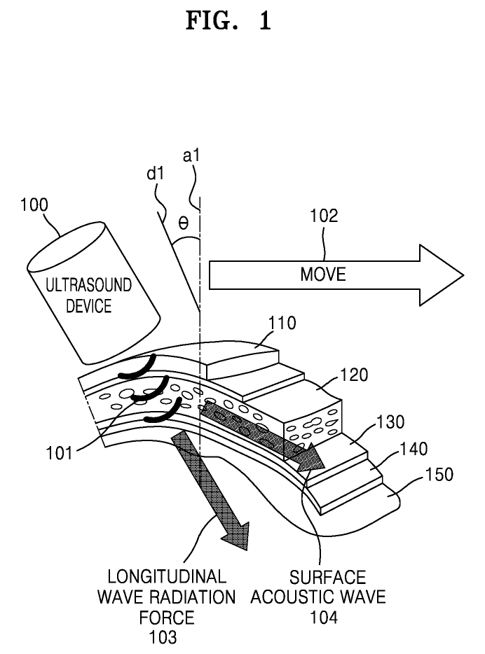

FIG. 1 is a diagram illustrating an operation concept of an ultrasound device 100 for accelerating lymphatic circulation according to an embodiment.

A method of radiating an ultrasound beam 101 for accelerating lymphatic circulation according to an embodiment may be performed by using the ultrasound device 100. The ultrasound device 100 may radiate the ultrasound beam 101 in a specific direction. The ultrasound device 100 may include an ultrasound converter to radiate the ultrasound beam 101 in a specific direction. For example, the ultrasound device device 100clude a mechanical or electronic ultrasound sweeper radiating the ultrasound beam 101. The ultrasound device 100 may radiate the ultrasound beam 101 while moving 102 a focusing point of the ultrasound beam 101. The ultrasound device 100 maydevice 100sly radiate the ultrasound beam 101.

The ultrasound device 100 may radiate the ultrasound beam 101 obliquely with respect to a vertical virtual axis. The vertical virtual axis may be a virtual axis perpendicular to a surface of a subject. For example, the vertical virtual axis may be a virtual axis perpendicular to a surface of at least one of a skin 110, a bone 120, a first membrane 130, a second membrane 140, and a third membrane 150. The vertical virtual axis may be a virtual axis perpendicular to the surface of the earth.

The ultrasound device 100 for accelerating lymphatic circulation may radiate the ultrasound beam 101 at a specified angle. The specified angle may be a preset angle for accelerating lymphatic circulation of the subject. An angle between the ultrasound beam 101 and the vertical virtual axis may be 25 degrees or more and 55 degrees or less. For example, when the ultrasound beam 101 is used to stimulate lymphatic vessels at the bottom of a skull, the ultrasound beam 101 may be radiated at an angle between 25 degrees and 55 degrees on an axis perpendicular to a skull surface.

The ultrasound beam 101 may be obliquely incident on the subject with respect to the vertical virtual axis. The ultrasound beam 101 may generate a longitudinal wave radiation force 103 and a surface acoustic wave 104 inside the subject. For example, the specified angle may be 25 degrees or more and 55 degrees or less.

The ultrasound beam 101 radiated from the ultrasound device 100 may be incident on the interior of the subject. The ultrasound beam 101 radiated from the ultrasound device 100 may be incident on the skin 110. The ultrasound beam 101 incident on the skin 110 may be incident on the bone 120. For example, when the subject is a brain, the bone 120 may be a skull. The ultrasound beam 101 incident on the bone 120 may be incident on the first membrane 130. For example, when the subject is a brain, the first membrane 130 may be a dura meter. The ultrasound beam 101 incident on the first membrane 130 may be incident on the second membrane 140. For example, when the subject is a brain, the second membrane 140 may be an arachnoid membrane. The ultrasound beam 101 incidenton the second membrane 140 may travel to the third membrane 150. For example, when the subject is a brain, the third membrane 150 may be a pia meter.

The longitudinal wave radiation force 103 may cause the ultrasound beam 101 to be incident on the skin 110, the bone 120, the first membrane 130, the second membrane 140, and the third membrane 150. The surface acoustic wave 104 may cause the incident ultrasound beam 101 to generate a transverse wave inside the skin 110, the bone 120, the first membrane 130, the second membrane 140, and the third membrane 150. The radiation force by the generated transverse wave and the incident longitudinal wave may accelerate lymphatic circulation in the subject. When lymphatic circulation of the subject is accelerated, waste materials inside the subject may be easily discharged.

The disclosure provides a technology for continuously radiating an ultrasound beam while moving an ultrasound focusing point. The ultrasound device 100 according to the disclosure may move 102 while radiating the ultrasound beam 101 to perform sweeping of the ultrasound beam 101 with respect to a target part.

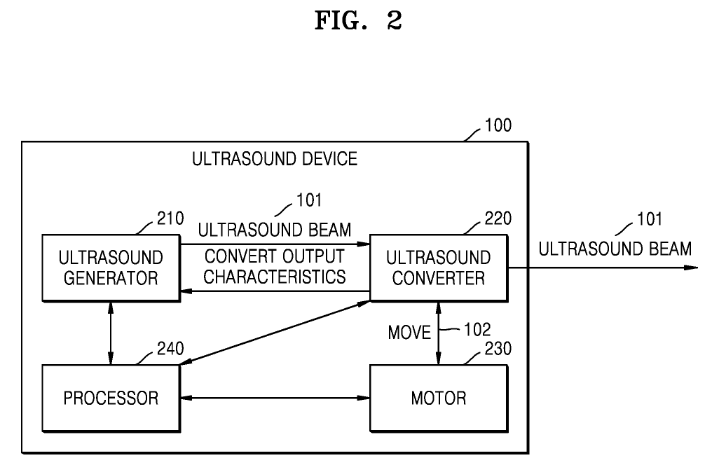

FIG. 2 is a block diagram illustrating an ultrasound device according to an embodiment. The ultrasound device 100 according to an embodiment may include an ultrasound generator 210, an ultrasound converter 220, a motor 230, and a processor 240.

The ultrasound generator 210 may generate the ultrasound beam 101. The ultrasound generator 210 may generate the ultrasound beam 101 based on set output characteristics. The output characteristics may include at least one of a width of the ultrasound beam 101, a moving speed of the ultrasound beam 101, and a scanning frequency of the ultrasound beam 101. The ultrasound generator 210 may transmit the generated ultrasound beam 101 to the ultrasound converter 220. The ultrasound generator 210 may have a single transmission channel.

The ultrasound converter 220 may receive the ultrasound beam 101 generated by the ultrasound generator 210. The ultrasound converter 220 may radiate the received ultrasound beam 101. The ultrasound converter 220 may radiate the ultrasound beam 101 to have a single focusing position. The ultrasound converter 220 may convert output characteristics of the ultrasound beam 101. The ultrasound converter 220 may transmit output characteristic conversion information to the ultrasound generator 210 to convert the output characteristics of the ultrasound beam 101.

The motor 230 may move 102 the ultrasound converter 220. The motor 230 may move 102 the ultrasound converter 220 so that the ultrasound converter 220 sweeps the subject.

The processor 240 may be operationally connected to the ultrasound generator 210, the ultrasound converter 220, and the motor 230. The processor 240 may include at least one processing circuit. The processor 240 may control the overall operations of the ultrasound generator 210, the ultrasound converter 220, and the motor 230.

The processor 240 may determine a time at which the ultrasound beam 101 is radiated from the ultrasound converter 220. The processor 240 may determine a time at which the ultrasound beam 101 needs to be radiated from the ultrasound device 100.

The processor 240 may control the ultrasound converter 220 to radiate the ultrasound beam 101 based on the time at which the ultrasound beam 101 is radiated. The processor 240 may inform the ultrasound converter 220 of the time at which the ultrasound beam 101 is radiated. The processor 240 may control the ultrasound converter 220 to radiate the ultrasound beam 101 at the determined time.

The processor 240 may move 102 the ultrasound converter 220 to correspond to a sweeping operation of the ultrasound beam 101 through the motor 230. The processor 240 may control the motor 230 to move 102 the ultrasound converter 220. The processor 240 may control the motor 230 to move 102 the ultrasound converter 220 so that the ultrasound beam 101 sweeps the subject. Accordingly, when radiating the ultrasound beam 101, the ultrasound device 100 according to an embodiment may move 102 in a direction in which the position of the ultrasound beam 101 is to be swept.

The processor 240 may convert the output characteristics of the ultrasound beam 101 to correspond to a focusing position of the ultrasound beam 101 during the sweeping operation through the ultrasound converter 220. The processor 240 may obtain information related to a position where the ultrasound beam 101 is focused when the ultrasound converter 220 radiates the ultrasound beam 101. The processor 240 may convert the output characteristics of the ultrasound beam 101 based on the information related to the position where the ultrasound beam 101 is focused.

The ultrasound device 100 according to an embodiment may be configured as a system including the single ultrasound generator 210, the single ultrasound converter 220, and the motor 230. The ultrasound device 100 according to an embodiment may continuously perform the sweeping operation through a mechanical movement. The ultrasound device 100 according to an embodiment may perform the sweeping operation at a desired speed through the mechanical movement.

For example, when the subject is a brain, the ultrasound device 100 according to an embodiment may measure the shape of the skull in a lower area of the ultrasound converter 220 based on CT, ultrasound, and an MRI image. The ultrasound device 100 according to an embodiment may predict a focusing point of the ultrasound beam 101 based on a measurement result. The ultrasound device 100 according to an embodiment may perform the sweeping operation through the ultrasound converter 220 and may move 102 the ultrasound beam 101. The ultrasound device 100 according to an embodiment may change the focusing position in real time. The ultrasound device 100 according to an embodiment may store the focusing point of the ultrasound beam 101 in a memory.

For example, when the subject is a brain, the ultrasound device 100 according to an embodiment may predict an area through which cerebrospinal fluid (CSF) may escape, including an area where a blood-brain barrier (BBB) is open, based on the MRI image. The ultrasound device 100 according to an embodiment may set the area through which the CSF may escape as a point where sweeping is performed. The ultrasound device 100 according to an embodiment may predict the point where sweeping is performed and store the predicted point in the memory. The ultrasound device 100 according to an embodiment may perform sweeping based on point information stored in the memory.

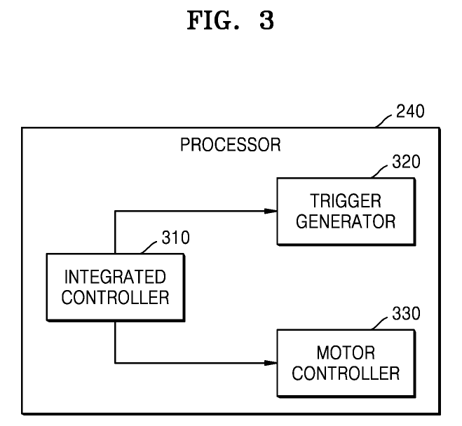

FIG. 3 is a block diagram illustrating the processor 240 according to an embodiment. The processor 240 according to an embodiment may include an integrated controller 310, a trigger generator 320, and a motor controller 330.

The integrated controller 310 may generate a control signal for controlling the motor 230 and a time signal including a time at which the ultrasound beam 101 is radiated. The integrated controller 310 may control a time at which the motor 230 moves. The integrated controller 310 may transmit, to the trigger generator 320, a time signal including a time at which the ultrasound beam 101 needs to be transmitted while the motor 230 is moving.

The trigger generator 320 may receive the time signal from the integrated controller 310 and generate a trigger signal for outputting the ultrasound beam 101 at the time based on the time signal. The trigger generator 320 may receive, from the integrated controller 310, the time signal including the time at which the ultrasound beam 101 needs to be transmitted. The trigger generator 320 may generate the trigger signal for controlling a waveform pattern of the ultrasound beam 101 so that a burst signal is repeatedly generated at each time included in the time signal.

The motor controller 330 may receive the control signal from the integrated controller 310 and generate a movement control signal for controlling the movement of the motor 230 based on the control signal. The motor controller 330 may generate the movement control signal by the control of the integrated controller 310 and transmit the generated movement control signal to a motor driver 450. For example, the motor controller 330 may generate a pulse width modulation (PWM) signal. The motor controller 330 may independently control the movement and speed of the motor 230 (e.g., a four-axis motor) having a plurality of drive shafts through the PWM signal.

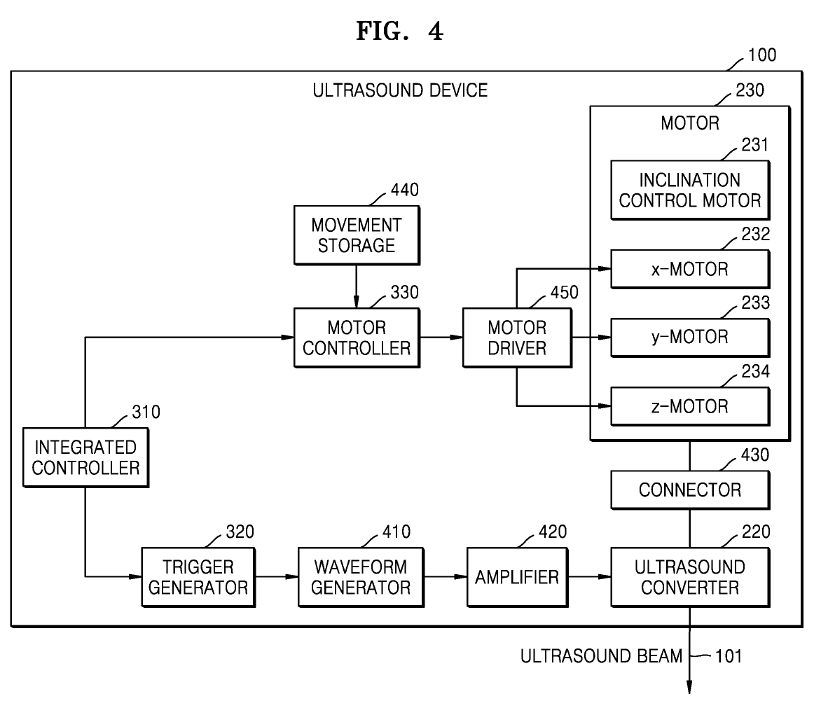

FIG. 4 is a block diagram illustrating the ultrasound device 100 according to an embodiment. The ultrasound device 100 according to an embodiment may further include a waveform generator 410, an amplifier 420, a connector 430, a movement storage 440, and the motor driver 450.

The waveform generator 410 may receive a trigger signal from the trigger generator 320 and generate a waveform of the ultrasound beam 101 based on the trigger signal. The waveform generator 410 may generate an ultrasound burst. The waveform generator 410 may generate a waveform of the ultrasound beam 101 by including the ultrasound burst in the ultrasound beam 101. The waveform generator 410 may be included in the ultrasound generator 210 (of FIG. 2).

The amplifier 420 may amplify the ultrasound beam 101 having the generated waveform and transmit the amplified ultrasound beam 101 to the ultrasound converter 220. The amplifier 420 may increase an amplitude of the waveform of the ultrasound beam 101. The amplifier 420 may transmit the ultrasound beam 101 with the increased amplitude to the ultrasound converter 220. The amplifier 420 may be included in the ultrasound generator 210.

The connector 430 may connect the motor 230 to the ultrasound converter 220. The connector 430 may cause the ultrasound converter 220 to move according to the movement of the motor 230.

The movement storage 440 may store information related to the movement of the motor 230. The movement storage 440 may store the movement of the ultrasound converter 220 over time. The movement storage 440 may transmit stored information to the motor controller 330.

The motor driver 450 may receive a movement control signal from the motor controller 330. The motor driver 450 may be driven based on the movement control signal. The motor driver 450 may control the motor 230 to move according to the information stored in the movement storage 440.

The motor 230 may include an inclination control motor 231, an x-motor 232, a y-motor 233, and a z-motor 234. The inclination control motor 231 may control a direction of the ultrasound converter 220. The x-motor 232 may move the ultrasound converter 220 in an x-axis direction by the control of the motor driver 450 driving based on the movement control signal. The y-motor 233 may move the ultrasound converter 220 in a y-axis direction by the control of the motor driver 450. The z-motor 234 may move the ultrasound converter 220 in a z-axis direction by the control of the motor driver 450. The motor 230 may move the ultrasound converter 220 through the connector 430.

The ultrasound converter 220 may radiate the ultrasound beam 101 while moving according to the movement of the motor 230. The ultrasound converter 220 may be connected to the motor 230 (e.g., a four-axis motor) having a plurality of drive shafts through the connector 430. An angle at which ultrasound converter 220 is driven may be changed according to the control of the motor 230. The ultrasound converter 220 may radiate the ultrasound beam 101 including a burst waveform at a constant pulse repetition frequency (PRF).

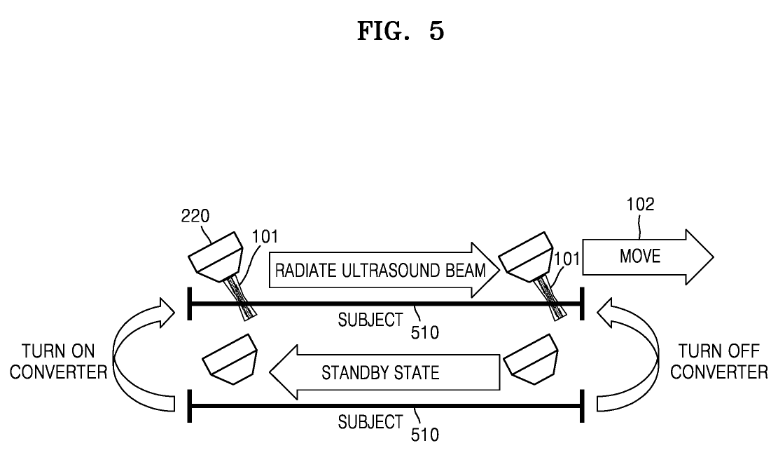

FIG. 5 is a diagram illustrating a method of operating the ultrasound converter 220 according to an embodiment.

The ultrasound converter 220 may be turned on to radiate the ultrasound beam 101 while moving 102 in a first direction during a sweeping operation. The first direction may be a direction in which the ultrasound beam 101 is to be swept to a subject 510. The ultrasound converter 220 may move 102 in the first direction while continuously radiating the ultrasound beam 101.

The ultrasound converter 220 may be turned off to maintain a standby state while moving in a second direction opposite to the first direction. The ultrasound converter 220 may move in the second direction and return to the original position. The ultrasound converter 220 may return to the original position while not radiating the ultrasound beam 101.

When performing the sweeping operation, the ultrasound converter 220 may radiate the ultrasound beam 101 only while moving 102 in the first direction to be swept, and may not radiate the ultrasound beam 101 when returning to the original position. Accordingly, the ultrasound converter 220 may sweep the ultrasound beam 101 in a desired direction. The ultrasound converter 220 may have at least one driving shaft. The ultrasound converter 220 may implement a reciprocating motion with respect to the driving shaft.

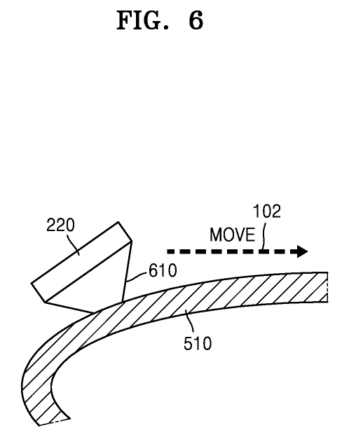

FIG. 6 is a diagram illustrating that the ultrasound converter 220 is in contact with the subject 510 according to an embodiment.

The ultrasound device 100 according to an embodiment may further include a beam path adjuster 610 in contact with a surface of the subject 510 from which the ultrasound beam 101 is radiated. The beam path adjuster 610 may be attached to the ultrasound converter 220. The beam path adjuster 610 may adjust a path of the ultrasound beam 101 such that the ultrasound beam 101 is incident on the subject 510. The beam path adjuster 610 may cause the ultrasound converter 220 to be in close contact with the surface of the subject 510. The beam path adjuster 610 may have a shape to be in close contact with the surface of the subject 510. For example, the beam path adjuster 610 may have a sloped shape.

The ultrasound converter 220 may be coupled to the beam path adjuster 610. The beam path adjuster 610 may be capable of adjusting an angle at which the ultrasound beam 101 is radiated. A tip portion of the ultrasound converter 220 may be in close contact with the surface of the subject 510 by using the beam path adjuster 610.

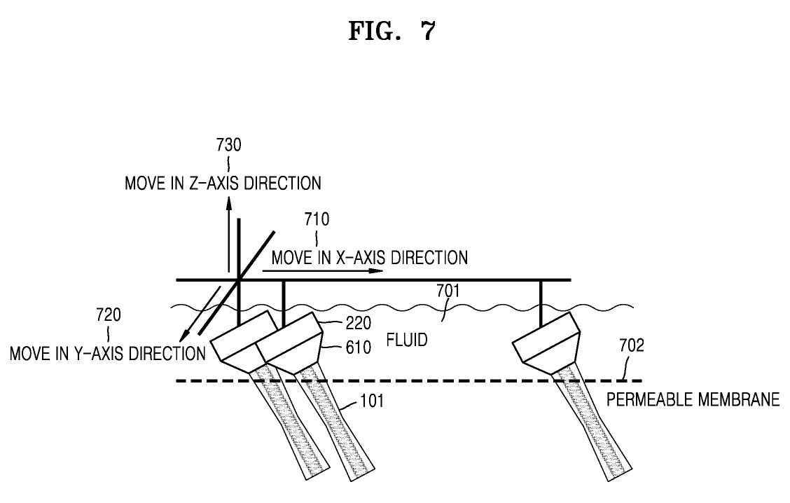

FIG. 7 is a diagram illustrating a method of moving the ultrasound converter 220 according to an embodiment.

The ultrasound converter 220 may be disposed inside a fluid 701. The fluid 701 may be surrounded by a permeable membrane 702. A converter module including the ultrasound converter 220 may be filled with the insulating fluid 701 such as silicone oil. A front portion of the ultrasound converter 220 may be a portion that radiates the ultrasound beam 101. In order to efficiently radiate the ultrasound beam 101, the front portion of the ultrasound converter 220 may be covered with a polymer film such as PET.

The ultrasound converter 220 may implement a three-dimensional sweeping operation through connection with a three-axis motor stage. The ultrasound converter 220 may perform a sweeping operation in an x-axis direction through an x-axis direction movement 710. The ultrasound converter 220 may perform the sweeping operation in a y-axis direction through a y-axis direction movement 720. The ultrasound converter 220 may perform the sweeping operation in a z-axis direction through a z-axis direction movement 730.

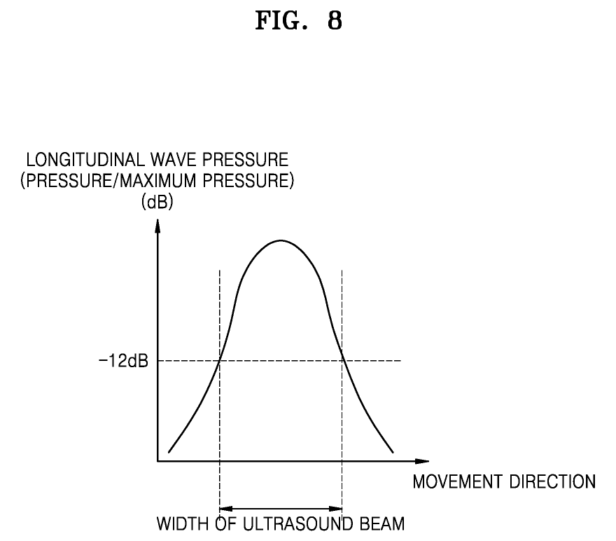

FIG. 8 is a graph illustrating a distribution of longitudinal wave pressure according to a moving direction of the ultrasound converter 220 according to an embodiment.

When the ultrasound converter 220 radiates the ultrasound beam 101, a radiation force may be applied to the subject 510. The radiation force applied to the subject 510 may be proportional to the longitudinal wave pressure applied by the ultrasound converter 220 in a vertical direction. The radiation force applied to the subject 510 may induce a movement to a direction in which the ultrasound converter 220 moves. A position to which the maximum ultrasound radiation force is applied may be changed according to the direction in which the ultrasound converter 220 moves. The graph of FIG. 8 shows the longitudinal wave pressure as a pressure relative to the maximum pressure in dB units. An area where a value of the longitudinal wave pressure is greater than or equal to a specified value may be set as a width of the ultrasound beam 101, and reflects the distribution of the radiation force. The specified value may be adjusted and changed. For example, an area where the longitudinal wave pressure is -12 dB or more relative to the maximum longitudinal wave pressure may be set as the width of the ultrasound beam 101.

In order to increase the directionality of moving waste materials and the sweeping efficiency during a sweeping operation of the ultrasound beam 220, the pressure distribution of the ultrasound beam 101 in the vertical direction (longitudinal direction) may be shown in a horizontal direction (transverse direction). A width of an area where the pressure of the ultrasound beam 101 relative to the maximum pressure in the vertical direction is greater than or equal to a specified value (e.g., -12 dB) may be defined as the width of the ultrasound beam 101.

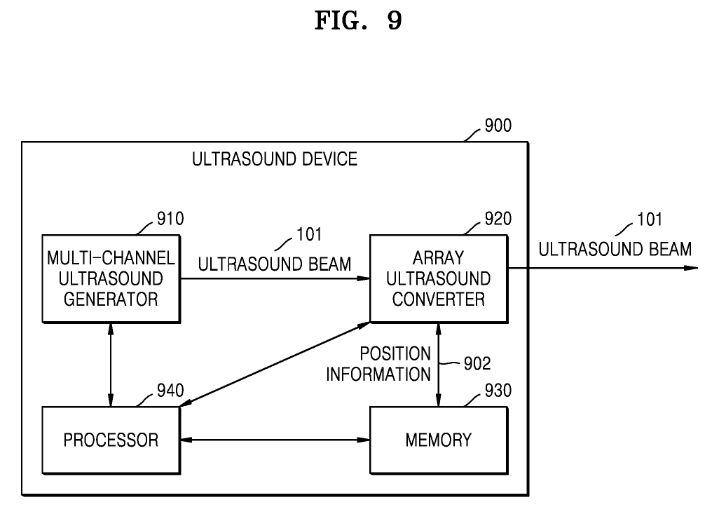

FIG. 9 is a block diagram illustrating an ultrasound device 900 according to an embodiment. The ultrasound device 900 according to an embodiment may include a multi-channel ultrasound generator 910, an array ultrasound converter 920, a memory 930, and a processor 940.

The multi-channel ultrasound generator 910 may include a plurality of channels. For example, the multi-channel ultrasound generator 910 may include 64 channels. The multi-channel ultrasound generator 910 may generate the ultrasound beam 101. The multi-channel ultrasound generator 910 may generate an ultrasound stimulation signal by activating at least one of the plurality of channels. The multi-channel ultrasound generator 910 may generate the ultrasound beam 101 by using the ultrasound stimulation signal. The multi-channel ultrasound generator 910 may transmit the generated ultrasound beam 101 to the ultrasound converter 220.

The array ultrasound converter 920 may radiate the ultrasound beam 101 generated by the multi-channel ultrasound generator 910 and simulate a movement of the ultrasound beam 101. The array ultrasound converter 920 may include a one-dimensional (1D) linear array or a two-dimensional (2D) array. The 1D linear array may include a lens in an elevational direction. The array ultrasound converter 920 may change a focusing position in a horizontal direction and a vertical direction by using the 2D array. The array ultrasound converter 920 may simulate a repetitive sweeping operation of the ultrasound beam 101. The array ultrasound converter 920 may simulate a reciprocating motion of the ultrasound beam 101. The array ultrasound converter 920 may simulate a three-dimensional (3D) sweeping motion by using the 2D array.

The memory 930 may store position information including a position at which the ultrasound beam 101 is radiated. The memory 930 may store the focusing position of the ultrasound beam 101. The memory 930 may store a part of a subject stimulated by the ultrasound beam 101.

The processor 940 may be operationally connected to the multi-channel ultrasound generator 910, the array ultrasound converter 920, and the memory 930. The processor 940 may include at least one processing circuit. The processor 940 may control the overall operations of the multi-channel ultrasound generator 910, the array ultrasound converter 920, and the memory 930.

The processor 940 may determine a time at which the ultrasound beam 101 is radiated from the array ultrasound converter 920. The processor 940 may determine a time at which the ultrasound beam 101 needs to be radiated from the ultrasound device 900. The processor 940 may include a timing controller adjusting a time at which the ultrasound beam 101 stimulates the subject.

The processor 940 may control the array ultrasound converter 920 to radiate the ultrasound beam 101 based on the time at which the ultrasound beam 101 is radiated. The processor 940 may notify the array ultrasound converter 920 of the time at which the ultrasound beam 101 is radiated. The processor 940 may control the array ultrasound converter 920 to radiate the ultrasound beam 101 at the determined time.

The processor 940 may move a focusing position of the ultrasound beam 101 to correspond to a sweeping operation of the ultrasound beam 101 or the position information stored in the memory 930 through the array ultrasound converter 920. The processor 940 may load a position for focusing the ultrasound beam 101 from the memory 930. The processor 940 may include a program for analyzing a position of a subject stimulated by the ultrasound beam 101.

The processor 940 may simulate the reciprocating motion of the ultrasound beam 101 to correspond to the focusing position of the ultrasound beam 101 during the sweeping operation through the array ultrasound converter 920. The processor 940 may perform ultrasound stimulation on the focusing position loaded from the memory 930.

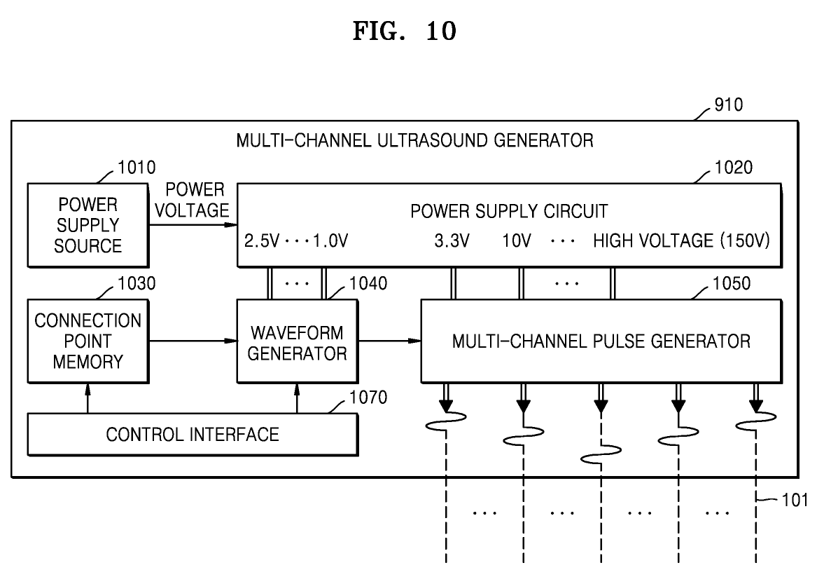

FIG. 10 is a block diagram illustrating the multi-channel ultrasound generator 910 according to an embodiment. The multi-channel ultrasound generator 910 according to an embodiment may include a power supply source 1010, a power supply circuit 1020, a focusing point memory 1030, a waveform generator 1040, a multi-channel pulse generator 1050, and a control info interface 1070.

The power supply source 1010 may supply a power supply voltage to the power supply circuit 1020. The power supply source 1010 may include an adapter for supplying the power supply voltage. The power supply voltage may have a voltage of specified magnitude. For example, the power supply voltage may be 24 V.

The power supply circuit 1020 may receive the power supply voltage from the power supply source 1010 and generate a plurality of voltage levels. The plurality of voltage levels may include 2.5 V, 1.0 V, 3.3 V, 10 V, and a high voltage (150 V). The power supply circuit 1020 may transmit the plurality of voltage levels to the waveform generator 1040 and the multi-channel pulse generator 1050.

The focusing point memory 1030 may store information related to a focusing point of the ultrasound beam 101. The focusing point memory 1030 may transmit the information related to the focusing point of the ultrasound beam 101 to the waveform generator 1040.

The waveform generator 1040 may receive the plurality of voltage levels and the information related to the focusing point of the ultrasound beam 101. The waveform generator 1040 may generate a waveform of the ultrasound beam 101 based on the plurality of voltage levels and the information related to the focusing point of the ultrasound beam 101. The waveform generator 1040 may transmit the generated waveform of the ultrasound beam 101 to the multi-channel pulse generator 1050.

The multi-channel pulse generator 1050 may receive the plurality of voltage levels and generate a plurality of pulses included in the ultrasound beam 101 based on the plurality of voltage levels. The multi-channel pulse generator 1050 may receive the waveform of the ultrasound beam 101 from the waveform generator 1040. The channel pulse generator 1050 may generate the ultrasound beam 101 to correspond to the received waveform of the transmitted ultrasound beam 101.

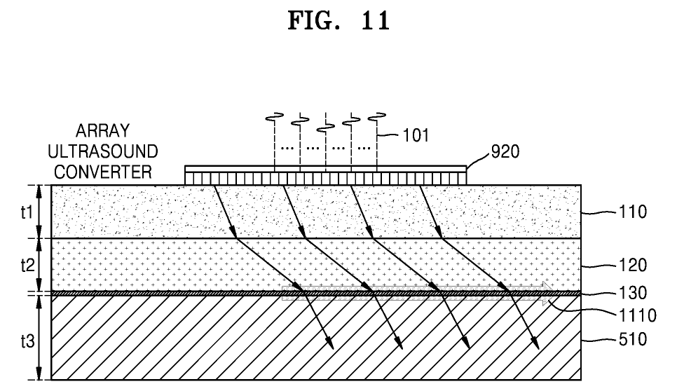

FIG. 11 is a diagram illustrating the array ultrasound converter 920 according to an embodiment.

The array ultrasound converter 920 may receive a plurality of pulses included in the ultrasound beam 101. The array ultrasound converter 920 may perform a sweeping 1110 operation of the ultrasound beam 101 based on the plurality of pulses.

The array ultrasound converter 920 may perform the sweeping 1110 operation on the skin 110 having a first thickness t1, the bone 120 having a second thickness t2, the first membrane 130, and the subject 510 having a third thickness t3.

The array ultrasound converter 920 may have a shape corresponding to a surface of the subject 510. For example, when the subject 510 is a brain, the array ultrasound converter 920 may have a bent shape to correspond to a surface shape of the skull.

The array ultrasound converter 920 may be in close contact with the surface of the subject 510. For example, an adhesive member (e.g., hydrogel) having an adhesion may be attached to a surface of the array ultrasound converter 920. In this case, the array ultrasound converter 920 may be in close contact with the surface of the skin 110.

The array ultrasound converter 920 may give an angle to the ultrasound beam 101 incident on the subject 510. The array ultrasound converter 920 may cause gaps between unit elements included in the array ultrasound converter 920 to be smaller than a transmission wavelength of the ultrasound beam 101 so as to reduce a grating lobe when giving the angle to the ultrasound beam 101.

The array ultrasound converter 920 may measure at least one of the first thickness t1 of the skin 110, the second thickness t2 of the bone 120, and the third thickness t3 of the subject 510. The array ultrasound converter 920 may adjust the focusing position of the ultrasound beam 101 and a pulse transmission time and a pulse transmission point of at least one of the plurality of channels for focusing the ultrasound beam 101 based on at least one of the first thickness t1 of the skin 110, the second thickness t2 of the bone 120, and the third thickness t3 of the subject 510.

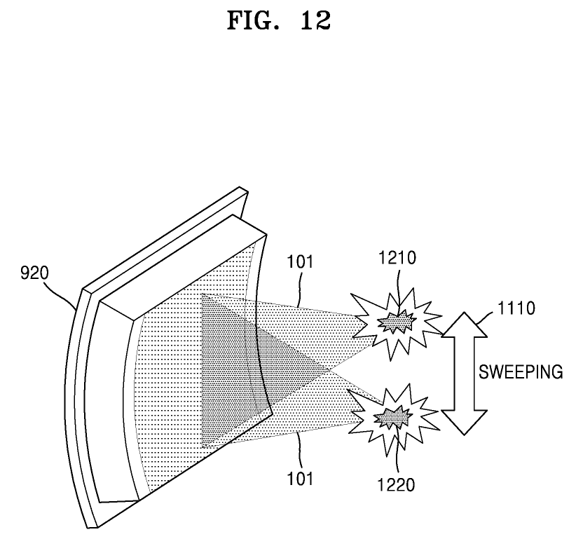

FIG. 12 is a diagram illustrating sweeping of the array ultrasound converter 920 according to an embodiment.

The array ultrasound converter 920 may perform the sweeping 1110 operation of the ultrasound beam 101 based on a plurality of pulses. The array ultrasound converter 920 may change a focusing point by adjusting a generation timing of the ultrasound beam 101 between a plurality of channels. The array ultrasound converter 920 may perform the sweeping 1110 operation between a first focusing point 1210 and a second focusing point 1220 by adjusting the generation timing of the ultrasound beam 101 between the plurality of channels.

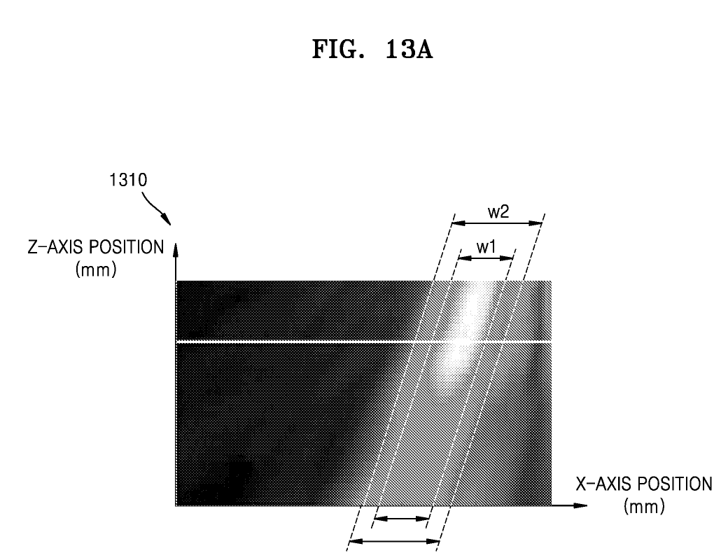

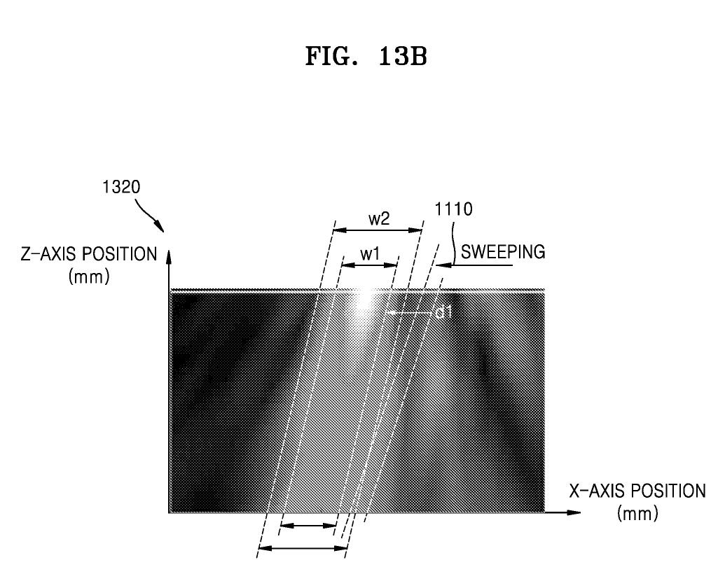

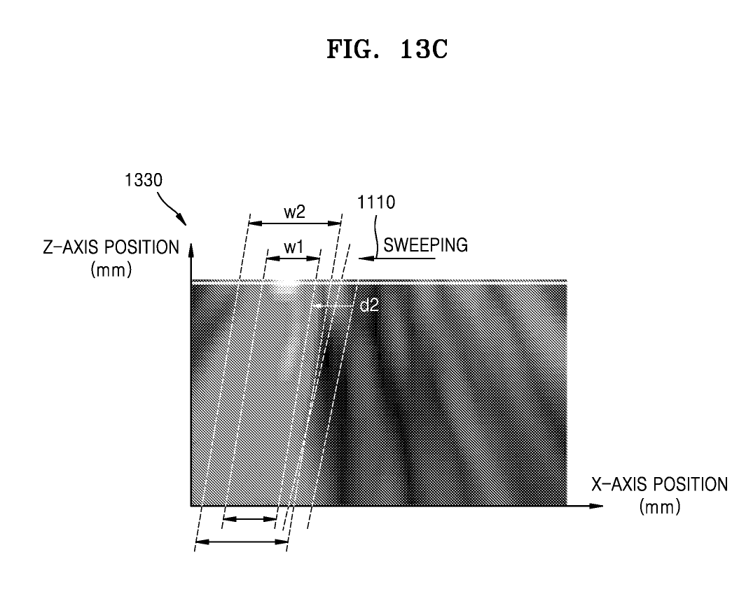

FIG. 13A is a diagram illustrating a sweeping form 1310 of the ultrasound device 900 according to an embodiment. FIG. 13B is a diagram illustrating a sweeping form 1320 of the ultrasound device 900 according to an embodiment. FIG. 13C is a diagram illustrating a sweeping form 1330 of the ultrasound device 900 according to an embodiment.

Each of movement distances d1 and d2 of the array ultrasound converter 920 at a position where the adjacent ultrasound beam 101 is scanned when the ultrasound beam 101 moves for the sweeping 1110 operation may be less than or equal to a first width w1, which is a width of a burst signal of the generated ultrasound beam 101. A second width W2, which is a width of a portion of the array ultrasound converter 920 that receives a longitudinal wave pressure by the adjacent ultrasound beam 101 when the ultrasound beam 101 moves for the sweeping 1110 operation may overlap at least partially. Accordingly, the ultrasound beam 101 scanned at the current position and the previous ultrasound beam 101 may spatially overlap at least partially. The processor 940 of the ultrasound device 900 may adjust a movement speed of the ultrasound beam 101 and a scanning frequency of the ultrasound beam 101 during the sweeping 1110 operation such that the ultrasound beam 101 scanned at the current position and the previous ultrasound beam 101 may spatially overlap at least partially.

Specifically, when the movement speed of the ultrasound beam 101 is v, the width of the ultrasound beam 101 is a, and a burst time interval of the ultrasound beam 101 is t, the ultrasound device 900 according to an embodiment needs to satisfy (v*t)<(a/2). The ultrasound device 900 according to an embodiment may a position change of the ultrasound beam 101 to be less than a/2 when the array ultrasound converter 920 irradiates the ultrasound beam 101 and then irradiates the next ultrasound beam 101.

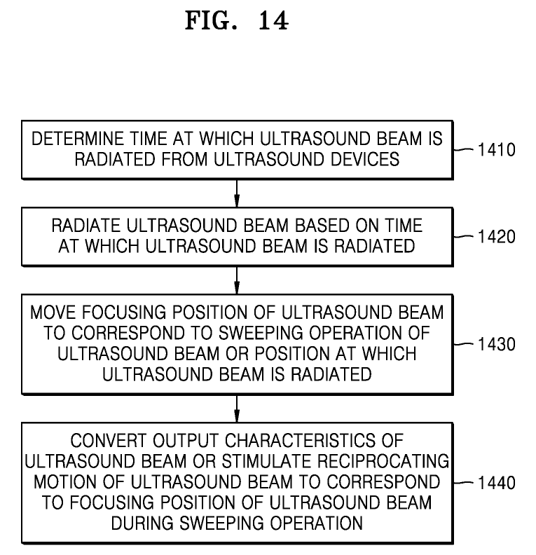

FIG. 14 is a flowchart illustrating a method of operating the ultrasound devices 100 and 900 according to an embodiment.

In operation 1410, a time at which an ultrasound beam is radiated from the ultrasound devices 100 and 900 may be determined through the ultrasound devices 100 and 900.

In operation 1420, the ultrasound beam may be radiated through the ultrasound devices 100 and 900based on the time at which the ultrasound beam is radiated.

In operation 1430, a focusing position of the ultrasound beam may be moved through the ultrasound devices 100 and 900 to correspond to a sweeping operation of the ultrasound beam or a position at which the ultrasound beam is radiated.

In operation 1440, output characteristics of the ultrasound beam may be converted or a reciprocating motion of the ultrasound beam may be simulated through the ultrasound devices 100 and 900 to correspond to the focusing position of the ultrasound beam during the sweeping operation.

The disclosure provides a technology for continuously radiating an ultrasound beam while moving an ultrasound focusing point.

An ultrasound device for accelerating lymphatic circulation according to an embodiment may include an ultrasound generator configured to generate an ultrasound beam, an ultrasound converter configured to radiate the ultrasound beam generated by the ultrasound generator and convert output characteristics of the ultrasound beam, a motor configured to move the ultrasound converter, and at least one processor operationally connected to the ultrasound generator, the ultrasound converter, and the motor, and the at least one processor may determine a time at which the ultrasound beam is radiated from the ultrasound converter, control the ultrasound converter to radiate the ultrasound beam based on the time at which the ultrasound beam is radiated, move the ultrasound converter to correspond to a sweeping operation of moving and radiating the ultrasound beam through the motor, and convert the output characteristics of the ultrasound beam through the ultrasound converter to correspond to a focusing position of the ultrasound beam during the sweeping operation.

In an embodiment, an angle between the ultrasound beam and a vertical virtual axis may be 25 degrees or more and 55 degrees or less.

In an embodiment, the output characteristics may include at least one of a width of the ultrasound beam, a movement speed of the ultrasound beam, and a scanning frequency of the ultrasound beam.

In an embodiment, the at least one processor may include an integrated controller configured to generate a control signal for controlling the motor and a time signal including the time at which the ultrasound beam is radiated, a trigger generator configured to receive the time signal from the integrated controller and generate a trigger signal for the ultrasound beam to be output at the time based on the time signal, and a motor controller configured to receive the control signal from the integrated controller and generate a movement control signal for controlling a movement of the motor based on the control signal.

In an embodiment, the ultrasound device may further include a waveform generator configured to receive the trigger signal from the trigger generator and generate a waveform of the ultrasound beam based on the trigger signal, and an amplifier configured to amplify the generated waveform and transmit the ultrasound beam from the ultrasound converter.

In an embodiment, the motor may include an inclination control motor configured to control a direction of the ultrasound converter, an x-motor configured to move the ultrasound converter in an x-axis direction by control of the motor driver driven based on the movement control signal, a y-motor configured to move the ultrasound converter in a y-axis direction by control of the motor driver, and a z-motor configured to move the ultrasound converter in a z-axis direction by control of the motor driver.

In an embodiment, the ultrasound converter may be turned on to radiate the ultrasound beam while moving in a first direction during the sweeping operation, and turned off to maintain a standby state while moving in a second direction opposite to the first direction.

In an embodiment, the ultrasound converter may include a beam path controller in contact with a surface of a subject from which the ultrasound beam is radiated.

An ultrasound device for accelerating lymphatic circulation according to an embodiment may include a multi-channel ultrasound generator configured to generate an ultrasound beam, an array ultrasound converter configured to radiate the ultrasound beam generated by the multi-channel ultrasound generator and simulate a movement of the ultrasound beam, a memory storing position information including a position at which the ultrasound beam is radiated, and at least one processor operationally connected to the multi-channel ultrasound generator, the array ultrasound converter, and the memory, and the at least one processor may determine a time at which the ultrasound beam is radiated from the array ultrasound converter, control the array ultrasound converter to radiate the ultrasound beam based on the time at which the ultrasound beam is radiated, move a focusing position of the ultrasound beam through the array ultrasound converter to correspond to a sweeping operation of the ultrasound beam or the position information stored in the memory, and simulate a reciprocating motion of the ultrasound beam through the array ultrasound converter to correspond to the focusing position of the ultrasound beam during the sweeping operation.

In an embodiment, an angle between the ultrasound beam and a vertical virtual axis may be 25 degrees or more and 55 degrees or less.

In an embodiment, the multi-channel ultrasound generator may include a power supply circuit configured to receive a power supply voltage from a power source and generate a plurality of voltage levels, and a multi-channel pulse generator configured to receive the plurality of voltage levels and generate a plurality of pulses included in the ultrasound beam based on the plurality of voltage levels.

In an embodiment, the array ultrasound converter may receive the plurality of pulses included in the ultrasound beam, and perform the sweeping operation of the ultrasound beam based on the plurality of pulses.

A method of operating an ultrasound device for accelerating lymphatic circulation according to an embodiment may include determining, by the ultrasound device, a time at which an ultrasound beam is radiated from the ultrasound device, radiating, by the ultrasound device, the ultrasound beam based on the time at which the ultrasound beam is radiated, moving, by the ultrasound device, a focusing position of the ultrasound beam to correspond to a sweeping operation of the ultrasound beam or a position at which the ultrasound beam is radiated, and converting, by the ultrasound device, output characteristics of the ultrasound beam or simulating a reciprocating motion of the ultrasound beam to correspond to the focusing position of the ultrasound beam during the sweeping operation.

In an embodiment, an angle between the ultrasound beam and a vertical virtual axis may be 25 degrees or more and 55 degrees or less.

According to the disclosure, the device continuously radiating an ultrasound beam while moving an ultrasound focusing point may be implemented.

The method according to an embodiment may be embodied as program instructions executable by various computer devices, and recorded on a computer-readable medium. The computer-readable medium may include a program command, a data file, a data structure, etc. alone or in combination. A program command recorded on the medium may be specially designed and configured for the disclosure or may be known to and usable by one of ordinary skill in the art of computer software. Examples of the computer-readable recording medium include magnetic media such as hard disks, floppy disks, or magnetic tapes, optical media such as compact disc ROMs (CD-ROMs) or digital video discs (DVDs), magneto-optical media such as floptical disks, and hardware devices such as ROM, RAM, and flash memory, which are specially configured to store and execute program instructions. An example of a program command may include not only machine codes created by a compiler, but also high-level programming language executable by a computer using an interpreter.

Some embodiments of the disclosure may also be implemented in the form of a recording medium including instructions executable by a computer, such as a program module executed by the computer. A computer-readable medium may be any available medium that may be accessed by a computer and includes both volatile and nonvolatile media and separable and non-separable media. In addition, the computer-readable medium may include both a computer storage medium and a communication medium. The computer-readable medium both volatile and nonvolatile media and separable and non-separable media implemented by any method or technique for storing information, such as computer-readable instructions, data structures, program modules, or other data. The communication medium typically includes computer-readable instructions, data structures, program modules, other data of modulated data signals such as carriers, or other transmission mechanisms, and includes any information transmission medium. In addition, some embodiments of the disclosure may be implemented as computer programs or computer program products that include instructions executable by a computer, such as a program module executed by the computer.

A device-readable storage medium may be provided in the form of a non-transitory storage medium. Here, the term 'non-transitory storage medium' means that it is a tangible device and does not include signals (e.g., electromagnetic waves), and this term does not distinguish between a case where data is stored semi-permanently in a storage medium and a case where data is temporarily stored. For example, the 'non-transitory storage medium' may include a buffer in which data is temporarily stored.

According to an embodiment, the method according to various embodiments disclosed in this document may be provided by being included in a computer program product. The computer program product may be traded between a seller and a buyer as commodity. The computer program product may be distributed in the form of a device-readable storage medium (e.g., compact disk read only memory (CD-ROM), or may be distributed online (e.g., downloaded or uploaded) directly through an application store or between two user devices (e.g., smartphones). In a case of online distribution, at least a part of the computer program product (e.g., a downloadable app) may be temporarily stored in or generated by a device-readable storage medium such as a server of a manufacturer, a server of an application store, or a memory of a relay server.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. An ultrasound device for accelerating lymphatic circulation, the ultrasound device comprising:
an ultrasound generator configured to generate an ultrasound beam;
an ultrasound converter configured to radiate the ultrasound beam generated by the ultrasound generator and convert output characteristics of the ultrasound beam;
a motor configured to move the ultrasound converter; and
at least one processor operationally connected to the ultrasound generator, the ultrasound converter, and the motor,
wherein the at least one processor is configured to
determine a time at which the ultrasound beam is radiated from the ultrasound converter,
control the ultrasound converter to radiate the ultrasound beam based on the time at which the ultrasound beam is radiated,
move the ultrasound converter to correspond to a sweeping operation of moving and radiating the ultrasound beam through the motor, and
convert the output characteristics of the ultrasound beam through the ultrasound converter to correspond to a focusing position of the ultrasound beam during the sweeping operation.

2. The ultrasound device of claim 1, wherein an angle between the ultrasound beam and a vertical virtual axis is 25 degrees or more and 55 degrees or less.

3. The ultrasound device of claim 1, wherein the output characteristics include at least one of a width of the ultrasound beam, a movement speed of the ultrasound beam, and a scanning frequency of the ultrasound beam.

4. The ultrasound device of claim 1, wherein
the at least one processor includes
an integrated controller configured to generate a control signal for controlling the motor and a time signal including the time at which the ultrasound beam is radiated;
a trigger generator configured to receive the time signal from the integrated controller and generate a trigger signal for the ultrasound beam to be output at the time based on the time signal; and
a motor controller configured to receive the control signal from the integrated controller and generate a movement control signal for controlling a movement of the motor based on the control signal.

5. The ultrasound device of claim 4, further comprising:
a waveform generator configured to receive the trigger signal from the trigger generator and generate a waveform of the ultrasound beam based on the trigger signal; and
an amplifier configured to amplify the generated waveform and transmit the ultrasound beam to the ultrasound converter.

6. The ultrasound device of claim 4, wherein the motor includes
an inclination control motor configured to control a direction of the ultrasound converter;
an x-motor configured to move the ultrasound converter in an x-axis direction by control of the motor driver driven based on the movement control signal;
a y-motor configured to move the ultrasound converter in a y-axis direction by control of the motor driver; and
a z-motor configured to move the ultrasound converter in a z-axis direction by control of the motor driver.

7. The ultrasound device of claim 1, wherein
the ultrasound converter is turned on to radiate the ultrasound beam while moving in a first direction during the sweeping operation, and turned off to maintain a standby state while moving in a second direction opposite to the first direction.

8. The ultrasound device of claim 1, wherein the ultrasound converter further includes a beam path controller in contact with a surface of a subject from which the ultrasound beam is radiated.

9. An ultrasound device for accelerating lymphatic circulation, the ultrasound device comprising:
a multi-channel ultrasound generator configured to generate an ultrasound beam;
an array ultrasound converter configured to radiate the ultrasound beam generated by the multi-channel ultrasound generator and simulate a movement of the ultrasound beam;
a memory storing position information including a position at which the ultrasound beam is radiated; and
at least one processor operationally connected to the multi-channel ultrasound generator, the array ultrasound converter, and the memory,
wherein the at least one processor is configured to
determine a time at which the ultrasound beam is radiated from the array ultrasound converter,
control the array ultrasound converter to radiate the ultrasound beam based on the time at which the ultrasound beam is radiated,
move a focusing position of the ultrasound beam through the array ultrasound converter to correspond to a sweeping operation of the ultrasound beam or the position information stored in the memory, and
simulate a reciprocating motion of the ultrasound beam through the array ultrasound converter to correspond to the focusing position of the ultrasound beam during the sweeping operation.

10. The ultrasound device of claim 9, wherein an angle between the ultrasound beam and a vertical virtual axis is 25 degrees or more and 55 degrees or less.

11. The ultrasound device of claim 9, wherein
the multi-channel ultrasound generator includes
a power supply circuit configured to receive a power supply voltage from a power source and generate a plurality of voltage levels; and
a multi-channel pulse generator configured to receive the plurality of voltage levels and generate a plurality of pulses included in the ultrasound beam based on the plurality of voltage levels.

12. The ultrasound device of claim 11, wherein
the array ultrasound converter is further configured to
receive the plurality of pulses included in the ultrasound beam, and
perform the sweeping operation of the ultrasound beam based on the plurality of pulses.

13. A method of operating an ultrasound device for accelerating lymphatic circulation, the method comprising:
determining, by the ultrasound device, a time at which an ultrasound beam is radiated from the ultrasound device;
radiating, by the ultrasound device, the ultrasound beam based on the time at which the ultrasound beam is radiated;
moving, by the ultrasound device, a focusing position of the ultrasound beam to correspond to a sweeping operation of the ultrasound beam or a position at which the ultrasound beam is radiated; and
converting, by the ultrasound device, output characteristics of the ultrasound beam or simulating a reciprocating motion of the ultrasound beam to correspond to the focusing position of the ultrasound beam during the sweeping operation.

14. The method of claim 13, wherein an angle between the ultrasound beam and a vertical virtual axis is 25 degrees or more and 55 degrees or less.
